(19) United States
(12) Patent Application Publication
(10) Pub. No.: US 2013/0109947 A1
(43) Pub. Date: May 2, 2013

(54) METHODS AND SYSTEMS FOR CONTINUOUS NON-INVASIVE BLOOD PRESSURE MEASUREMENT USING PHOTOACOUSTICS

(71) Applicant: Lockett Wood, Lyons, CO (US)

(72) Inventor: Lockett Wood, Lyons, CO (US)

(73) Assignee: Nellcor Puritan Bennett LLC, Boulder, CO (US)

(21) Appl. No.: 13/284,226

(22) Filed: Oct. 28, 2011

(51) Int. Cl. A61B 6/00

(52) U.S. Cl. 600/407

(57) ABSTRACT

A patient monitoring system may use photoacoustic sensing to determine one or more physiological parameters of a subject. The system may detect an acoustic pressure response generated by the application and absorption of light, which may be used to identify the size of a blood vessel. The blood pressure of a subject may calculated based on the measured blood vessel size and calibration values. One or more calibration values may be determined based on a known relationship between a change in blood vessel size and a change in blood pressure.

The present disclosure relates to measuring continuous non-invasive blood pressure using photoacoustics.

### SUMMARY

A photoacoustic system may supply light energy to a region of interest such that the region of interest emits an acoustic pressure signal. The acoustic pressure signal may be analyzed to reveal features below the human skin such as the dimensions of a blood vessel. Photoacoustic techniques may be capable of operating at a resolution and frequency necessary to detect changes in blood vessel size over a cardiac cycle.

A blood vessel may stretch in response to the normal changes in blood pressure that occur during a cardiac cycle. Distensibility and compliance are example physiological features that quantify the response of the blood vessel to changes in blood pressure. Distensibility and compliance may be different in individuals, and may change for individuals with age or other changes in physical condition.

Calibration values may be calculated based on a known relationship between blood vessel size and blood pressure. Calibration values may be calculated for particular individuals, e.g., by measuring a change in blood vessel size while measuring blood pressure. Calibration values may also be determined based on information about the subject such as age, height, weight, physical condition, and medical history. Based on the calibration values for a patient, a photoacoustic system may non-invasively monitor the change in the blood vessel size over time to determine blood pressure.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system, in accordance with some embodiments of the present disclosure;

FIG. 2 is a block diagram of the illustrative patient monitoring system of FIG. 1 coupled to a subject, in accordance with some embodiments of the present disclosure;

FIG. 3 shows a block diagram of an illustrative signal processing system, in accordance with some embodiments of the present disclosure;

FIG. 4 is an illustrative photoacoustic arrangement, in accordance with some embodiments of the present disclosure;

FIG. 5 is a plot of an illustrative photoacoustic signal, including peaks corresponding to walls of blood vessels, in accordance with some embodiments of the present disclosure;

FIG. 6 is a flow diagram of illustrative steps for determining calibration values for a subject, in accordance with some embodiments of the present disclosure;

FIG. 7 is a plot of determining calibration values based on measured calibration points for two subjects; and

FIG. 8 is a flow diagram of illustrative steps for determining blood pressure based on photoacoustic measurement of a blood vessel, in accordance with some embodiments of the present disclosure.

### DETAILED DESCRIPTION OF THE FIGURES

Photoacoustics or the photoacoustic effect refers to the phenomenon in which one or more wavelengths of light are presented to and absorbed by one or more constituents of an object, thereby causing an increase in kinetic energy of the one or more constituents, which causes an associated pressure response within the object. Particular modulations or pulsing of the incident light, along with measurements of the corresponding pressure response in, for example, tissue of the subject, may be used for medical imaging, physiological parameter determination, or both. For example, the blood pressure of a subject may be determined using photoacoustic analysis.

A photoacoustic system may include a photoacoustic sensor that is placed at a site on a subject, typically a wrist, palm, elbow, neck, forehead, temple, or other location where blood vessels are within the sensitivity range of the instrument. The photoacoustic system may use a light source, and any suitable light guides (e.g., fiber optics), to pass light through the subject's tissue, or a combination of tissue thereof (e.g., organs), and an acoustic detector to sense the pressure response of the tissue. Tissue may include muscle, fat, blood, blood vessels, and/or any other suitable tissue types. In some embodiments, the light source may be a laser or laser diode, operated in pulsed or continuous wave (CW) mode. In some embodiments, the acoustic detector may be an ultrasound detector, which may be suitable to detect pressure fluctuations arising from the constituent's response to the incident light of the light source.

In some embodiments, the light from the light source may be focused, shaped, or otherwise spatially modulated to illuminate a particular region of interest. In some arrangements, photoacoustic monitoring may allow relatively higher spatial resolution than line of sight optical techniques (e.g., path integrated absorption measurements). The enhanced spatial resolution of the photoacoustic technique may allow for imaging, scalar field mapping, and other spatially resolved results, in 1, 2, or 3 spatial dimensions. The acoustic response to the photonic excitation may radiate from the illuminated region of interest, and accordingly may be detected at multiple positions.

The photoacoustic system may measure the pressure response that is received at the acoustic sensor as a function of time. The photoacoustic system may also include sensors at multiple locations. A signal representing pressure versus time or a mathematical manipulation of this signal (e.g., a scaled version thereof, etc.) may be referred to as the photoacoustic (PA) signal. The PA signal may be used to calculate any of a number of physiological parameters, including blood pressure. In some embodiments, PA signals from multiple spatial locations may be used to construct an image (e.g., imaging blood vessels).

In some applications, the light passed through the tissue is selected to be of one or more wavelengths that are absorbed by the constituent in an amount representative of the amount of the constituent present in the tissue. The absorption of light passed through the tissue varies in accordance with the amount of the constituent in the tissue. For example, for determining blood pressure, an infrared (IR) wavelength, for example 795 or 808 nm, may be used because it is sufficiently absorbed by blood. If additional physiological parameters are also determined using the photoacoustic system (e.g., oxygen saturation), Red and IR wavelengths may be used because highly oxygenated blood will absorb relatively less Red light and more IR light than blood with a lower oxygen saturation.

Any suitable light source may be used, and characteristics of the light provided by the light source may be controlled in any suitable manner. In some embodiments, a pulsed light source may be used to provide relatively short-duration pulses (e.g., nanosecond pulses) of light to the region of interest. Accordingly, the use of a pulse light source may result in a relatively broadband acoustic response (e.g., depending on the pulse duration). The use of a pulsed light source will be referred to herein as the “Time Domain Photoacoustic” (TD-PA) technique. A convenient starting point for analyzing a TD-PA signal is given by Eq. 1:

p(z)=Γμₐφ(z) (1)

under conditions where the irradiation time is small compared to the characteristic thermal diffusion time such as produced by a short-duration pulsed light source. Referring to Eq. 1, p(z) is the PA signal (indicative of the maximum induced pressure rise) at spatial location z indicative of acoustic pressure, Γ is the dimensionless Grüneisen parameter of the tissue, μₐ is the effective absorption coefficient of the tissue (or constituent thereof) to the incident light, and φ(z) is the optical fluence at spatial location z. The Grüneisen parameter is a dimensionless description of thermoelastic effects, and may be illustratively formulated by Eq. 2:

$$Γ = \frac{β {c}_{a}^{2}}{{C}_{p}} \qquad ( 2 )$$

where cₐ² is the speed of sound in the tissue, β is the isobaric volume thermal expansion coefficient, and Cₚ is the specific heat at constant pressure. In some circumstances, the optical fluence, at spatial location z (within the subject's tissue) of interest may be dependent upon the light source, the location itself (e.g., the depth), and optical properties (e.g., scattering coefficient, absorption coefficient, or other properties) along the optical path. For example, Eq. 3 provides an illustrative expression for the attenuated optical fluence at a depth z:

φ(z)=φ₀e^{μ}^{_{eff}}^{z} (3)

where φ₀ is the optical fluence from the light source incident at the tissue surface, z is the path length (i.e., the depth into the tissue in this example), and μ_{eff} is an effective attenuation coefficient of the tissue along the path length in the tissue in this example.

In some embodiments, a more detailed expression or model may be used rather than the illustrative expression of Eq. 3. In some embodiments, the actual pressure encountered by an acoustic detector may be proportional to Eq. 1, as the focal distance and solid angle (e.g., face area) of the detector may affect the actual measured PA signal. In some embodiments, an ultrasound detector positioned relatively farther away from the region of interest, will encounter a relatively smaller acoustic pressure. For example, the acoustic pressure received at a circular area A_{d} positioned at a distance R from the illuminated region of interest may be given by Eq. 4:

P_{d}=p(z)f(rₛ,R,A_{d}) (4)

where rₛ is the radius of the illuminated region of interest (and typically rₛ<R), and p(z) is given by Eq. 1. In some embodiments, the detected acoustic pressure amplitude may decrease as the distance R increases (e.g., for a spherical acoustic wave).

In some embodiments, a modulated CW light source may be used to provide a photonic excitation of a tissue constituent to cause a photoacoustic response in the tissue. The CW light source may be intensity modulated at one or more characteristic frequencies. The use of a CW light source, intensity modulated at one or more frequencies, will be referred to herein as the “Frequency Domain Photoacoustic” (FD-PA) technique. Although the FD-PA technique may include using frequency domain analysis, the technique may use time domain analysis, wavelet domain analysis, or any other suitable analysis, or any combination thereof. Accordingly, the term “frequency domain” as used in “FD-PA” refers to the frequency modulation of the photonic signal, and not to the type of analysis used to process the photoacoustic response.

Under some conditions, the acoustic pressure p(R,t) at detector position R at time t, may be shown illustratively by Eq. 5:

$$p ( R , t ) ∼ \frac{{p}_{0} ( {r}_{0} , ω )}{R} {e}^{- i ω ( t - τ )} \qquad ( 5 )$$

where r₀ is the position of the illuminated region of interest, ω is the angular frequency of the acoustic wave (caused by modulation of the photonic signal at frequency ω), R is the distance between the illuminated region of interest and the detector, and τ is the travel time delay of the wave equal to R/cₐ, where cₐ is the speed of sound in the tissue. The FD-PA spectrum p₀(r₀,ω) of acoustic waves is shown illustratively by Eq. 6:

$${p}_{0} ( {r}_{0} , ω ) = \frac{{Γμ}_{a} φ ( {r}_{0} )}{2 ( {μ}_{a} {c}_{a} - i ω )} \qquad ( 6 )$$

where μₐ cₐ, represents a characteristic frequency (and corresponding time scale) of the tissue.

In some embodiments, a FD-PA system may temporally vary the characteristic modulation frequency of the CW light source, and accordingly the characteristic frequency of the associated acoustic response. For example, the FD-PA system may use linear frequency modulation (LFM), either increasing or decreasing with time, which is sometimes referred to as “chirp” signal modulation. Shown in Eq. 7 is an illustrative expression for a sinusoidal chirp signal r(t):

$$r ( t ) = sin ( t ( {ω}_{0} + \frac{b}{2} t ) ) \qquad ( 7 )$$

where ω₀ is a starting angular frequency, and b is the angular frequency scan rate. Any suitable range of frequencies (and corresponding angular frequencies) may be used for modulation such as, for example, 1-5 MHz, 200-800 kHz, or other suitable range, in accordance with present disclosure. In some embodiments, signals having a characteristic frequency that changes as a nonlinear function of time may be used. Any suitable technique, or combination of techniques thereof, may be used to analyze a FD-PA signal. Two such exemplary techniques, a correlation technique and a heterodyne mixing technique, will be discussed below as illustrative examples.

In some embodiments, the correlation technique may be used to determine the travel time delay of the FD-PA signal. In some embodiments, a matched filtering technique may be used to process a PA signal. As shown in Eq. 8:

$${B}_{s} ( t - τ ) = \frac{1}{2 π} {\int }_{- ∞}^{∞} H ( ω ) S ( ω ) {e}^{i ω t} d w \qquad ( 8 )$$

Fourier transforms (and inverse transforms) are used to calculate the filter output Bₛ(t−T), in which H(ω) is the filter frequency response, S(ω) is the Fourier transform of the PA signal s(t), and T is the phase difference between the filter and signal. In some circumstances, the filter output of expression of Eq. 8 may be equivalent to an autocorrelation function. Shown in Eq. 9:

$$S ( ω ) = \frac{1}{2 π} {\int }_{- ∞}^{∞} s ( t ) {e}^{- i ω t} d t \qquad ( 9 )$$

is an expression for computing the Fourier transform S(ω) of the PA signal s(t). Shown in Eq. 10:

H(ω)=S*(ω)e^{-iωτ} (10)

is an expression for computing the filter frequency response H(ω) based on the Fourier transform of the PA signal s(t). It can be observed that the filter frequency response of Eq. 10 requires the frequency character of the PA signal be known beforehand to determine the frequency response of the filter. In some embodiments, as shown by Eq. 11:

$$B ( t ) = {\int }_{- ∞}^{∞} r ( {t}^{′} ) s ( t + {t}^{′} ) d {t}^{′} \qquad ( 11 )$$

the known modulation signal r(t) may be used for generating a cross-correlation with the PA signal. The cross-correlation output B(t) of Eq. 11 is expected to exhibit a peak at a time t equal to the acoustic signal travel time τ. Assuming that the temperature response and resulting acoustic response follow the illumination modulation (e.g., are coherent), Eq. 11 may allow calculation of the time delay, depth information, or both.

In some embodiments, the heterodyne mixing technique may be used to determine the travel time delay of the FD-PA signal. The ED-PA signal, as described above, may have similar frequency character as the modulation signal (e.g., coherence), albeit shifted in time due to the travel time of the acoustic signal. For example, a chirp modulation signal, such as r(t) of Eq. 7, may be used to modulate a CW light source. Heterodyne mixing uses the trigonometric identity of the following Eq. 12:

sin(A)sin(B)=½[cos(A−B)−cos(A+B)] (12)

which shows that two signals may be combined by multiplication to give periodic signals at two distinct frequencies (i.e., the sum and the difference of the original frequencies). If the result is passed through a low-pass filter to remove the higher frequency term (i.e., the sum), the resulting filtered, frequency shifted signal may be analyzed. For example, Eq. 13 shows a heterodyne signal L(t):

$$\begin{matrix}L ( t ) = 〈 r ( t ) s ( t ) 〉 \\ ≅ 〈 K r ( t ) r ( t - \frac{R}{{c}_{a}} ) 〉 \\ = \frac{1}{2} K cos ( \frac{R}{{c}_{a}} b t + θ )\end{matrix} \qquad ( 13 )$$

calculated by low-pass filtering (shown by angle brackets) the product of modulation signal r(t) and PA signal s(t). If the PA signal is assumed to be equivalent to the modulation signal, with a time lag R/cₐ due to travel time of the acoustic wave and amplitude scaling K, then a convenient approximation of Eq. 13 may be made, giving the rightmost term of Eq. 13. Analysis of the rightmost expression of Eq. 13 may provide depth information, travel time, or both. For example, a fast Fourier transform (FFT) may be performed on the heterodyne signal, and the frequency associated with the highest peak may be considered equivalent to time lag Rb/cₐ. Assuming that the frequency scan rate b and the speed of sound cₐ are known, the depth R may be estimated.

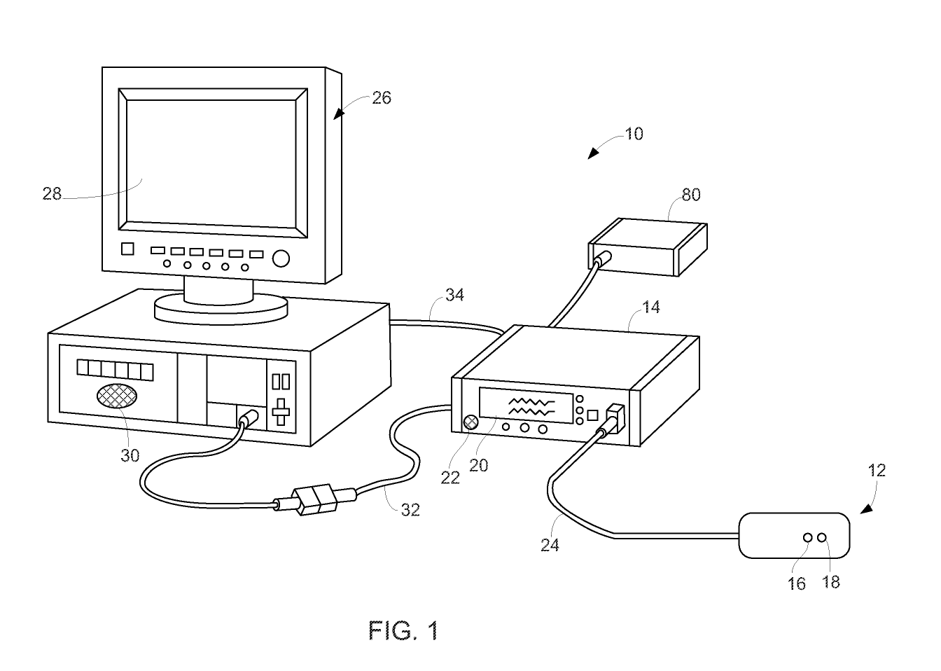

FIG. 1 is a perspective view of an embodiment of a physiological monitoring system 10. System 10 may include sensor unit 12 and monitor 14. In some embodiments, sensor unit 12 may be part of a photoacoustic monitor or imaging system. Sensor unit 12 may include a light source 16 for emitting light at one or more wavelengths into a subject's tissue. A detector 18 may also be provided in sensor unit 12 for detecting the acoustic (e.g., ultrasound) response that travels through the subject's tissue. Any suitable physical configuration of light source 16 and detector 18 may be used. In an embodiment, sensor unit 12 may include multiple light sources and/or acoustic detectors, which may be spaced apart. System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12 (e.g., a photoplethysmograph sensor). Multiple sensor units may be capable of being positioned at two different locations on a subject's body; for example, a first sensor unit may be positioned on a subject's forehead, while a second sensor unit may be positioned at a subject's fingertip.

In some embodiments, system 10 may include two or more sensors forming a sensor array in lieu of either or both of the sensor units. In some embodiments, a sensor array may include multiple light sources, detectors, or both. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data relating to light emission and acoustic detection received from one or more sensor units such as sensor unit 12. For example, monitor 14 may be configured to determine blood pressure, pulse rate, blood oxygen saturation (e.g., arterial, venous, or both), hemoglobin concentration (e.g., oxygenated, deoxygenated, and/or total), any other suitable physiological parameters, or any combination thereof. In some embodiments, calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a subject's physiological parameters are not within a predefined normal range. In some embodiments, the system 10 includes a stand-alone monitor in communication with the monitor 14 via a cable or a wireless network link.

In some embodiments, sensor unit 12 may be communicatively coupled to monitor 14 via a cable 24. Cable 24 may include electronic conductors (e.g., wires for transmitting electronic signals from detector 18), optical fibers (e.g., multi-mode or single-mode fibers for transmitting emitted light from light source 16), any other suitable components, any suitable insulation or sheathing, or any combination thereof. In some embodiments, a wireless transmission device (not shown) or the like may be used instead of or in addition to cable 24. Monitor 14 may include a sensor interface configured to receive physiological signals from sensor unit 12, provide signals and power to sensor unit 12, or otherwise communicate with sensor unit 12. The sensor interface may include any suitable hardware, software, or both, which may allow communication between monitor 14 and sensor unit 12.

In the illustrated embodiment, system 10 includes a multi-parameter physiological monitor 26. The monitor 26 may include a cathode ray tube display, a flat panel display (as shown) such as a liquid crystal display (LCD) or a plasma display, or may include any other type of monitor now known or later developed. Multi-parameter physiological monitor 26 may be configured to calculate physiological parameters and to provide a display 28 for information from monitor 14 and from other medical monitoring devices or systems (not shown). For example, multi-parameter physiological monitor 26 may be configured to display an estimate of a subject's blood pressure, blood oxygen saturation, hemoglobin concentration, and/or pulse rate generated by monitor 14. Multi-parameter physiological monitor 26 may include a speaker 30.

Monitor 14 may be communicatively coupled to multi-parameter physiological monitor 26 via a cable 32 or 34 that is coupled to a sensor input port or a digital communications port, respectively and/or may communicate wirelessly (not shown). In addition, monitor 14 and/or multi-parameter physiological monitor 26 may be coupled to a network to enable the sharing of information with servers or other workstations (not shown). Monitor 14 may be powered by a battery (not shown) or by a conventional power source such as a wall outlet.

Calibration device 80, which may be powered by monitor 14, a battery, or by a conventional power source such as a wall outlet, may include any suitable blood pressure calibration device. For example, calibration device 80 may take the form of any invasive or non-invasive blood pressure monitoring or measuring system used to generate reference blood pressure measurements for use in calibrating the blood pressure monitoring techniques described herein. Such calibration devices may include, for example, an aneroid or mercury sphygmomanometer and occluding cuff, a pressure sensor inserted directly into a suitable artery of a patient, an oscillometric device or any other device or mechanism used to sense, measure, determine, or derive a reference blood pressure measurement. In some embodiments, calibration device 80 may include a manual input device (not shown) used by an operator to manually input reference blood pressure measurements obtained from some other source (e.g., an external invasive or non-invasive blood pressure measurement system).

Calibration device 80 may also access reference blood pressure measurements stored in memory (e.g., RAM, ROM, or a storage device). For example, in some embodiments, calibration device 80 may access reference blood pressure measurements from a relational database stored within calibration device 80, monitor 14, or multi-parameter patient monitor 26. As described in more detail below, the reference blood pressure measurements generated or accessed by calibration device 80 may be updated in real-time, resulting in a continuous source of reference blood pressure measurements for use in continuous or periodic calibration. Alternatively, reference blood pressure measurements generated or accessed by calibration device 80 may be updated periodically, and calibration may be performed on the same periodic cycle. In the depicted embodiments, calibration device 80 is connected to monitor 14 via cable 82. In other embodiments, calibration device 80 may be a stand-alone device that may be in wireless communication with monitor 14. Reference blood pressure measurements may then be wirelessly transmitted to monitor 14 for use in calibration. In still other embodiments, calibration device 80 is completely integrated within monitor 14.

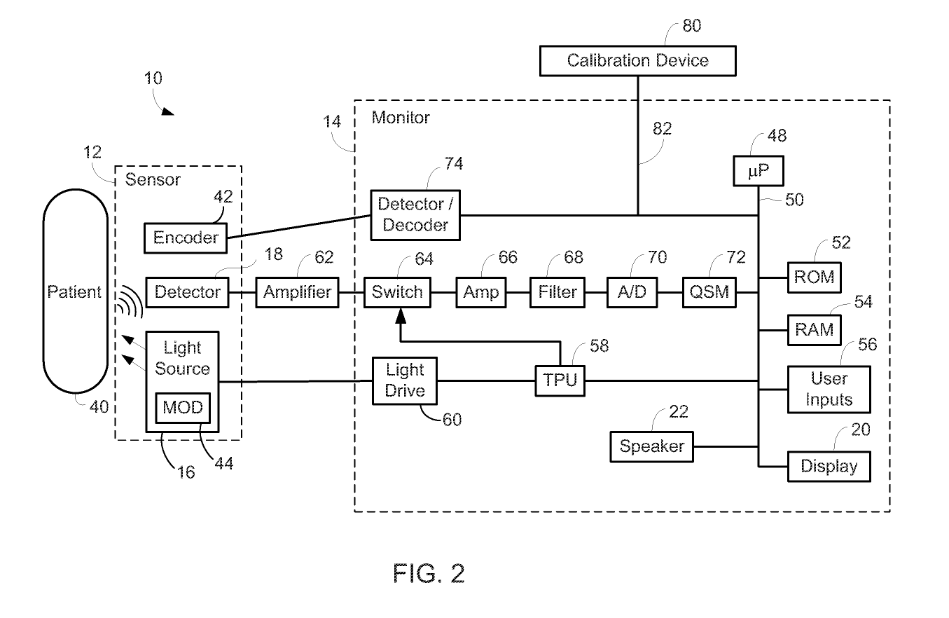

FIG. 2 is a block diagram of a physiological monitoring system, such as physiological monitoring system 10 of FIG. 1, which may be coupled to a subject 40 in accordance with an embodiment. Certain illustrative components of sensor unit 12 and monitor 14 are illustrated in FIG. 2.

Sensor unit 12 may include light source 16, detector 18, and encoder 42. In some embodiments, light source 16 may be configured to emit one or more wavelengths of light (e.g., visible, infrared) into a subject's tissue 40. Hence, light source 16 may provide Red light, IR light, any other suitable light, or any combination thereof, that may be used to calculate the subject's physiological parameters. In some embodiments, the Red wavelength may be between about 600 nm and about 700 nm, and the IR wavelength may be between about 800 nm and about 1000 nm. In embodiments where a sensor array is used in place of a single sensor, each sensor may be configured to provide light of a single wavelength. For example, a first sensor may emit only a Red light while a second may emit only an IR light. In a further example, the wavelengths of light used may be selected based on the specific location of the sensor.

It will be understood that, as used herein, the term “light” may refer to energy produced by electromagnetic radiation sources. Light may be of any suitable wavelength and intensity, and modulations thereof, in any suitable shape and direction. Detector 18 may be chosen to be specifically sensitive to the acoustic response of the subject's tissue arising from use of light source 16. It will also be understood that, as used herein, the “acoustic response” may refer to pressure and changes thereof caused by a thermal response (e.g., expansion and contraction) of tissue to light absorption by the tissue or constituent thereof.

In some embodiments, detector 18 may be configured to detect the acoustic response of tissue to the photonic excitation caused by the light source. In some embodiments, detector 18 may be a piezoelectric transducer which may detect force and pressure and output an electrical signal via the piezoelectric effect. In some embodiments, detector 18 may be a Faby-Pérot interferometer, or etalon. For example, a thin film (e.g., composed of a polymer) may be irradiated with reference light, which may be internally reflected by the film. Pressure fluctuations may modulate the film thickness, thus causing changes in the reference light reflection which may be measured and correlated with the acoustic pressure. In some embodiments, detector 18 may be configured or otherwise tuned to detect acoustic response in a particular frequency range. Detector 18 may convert the acoustic pressure signal into an electrical signal (e.g., using a piezoelectric material, photodetector of a Faby-Perot interferometer, or other suitable device). After converting the received acoustic pressure signal to an electrical signal, detector 18 may send the signal to monitor 14, where physiological parameters may be calculated based on the photoacoustic activity within the subject's tissue 40.

In some embodiments, encoder 42 may contain information about sensor unit 12, such as what type of sensor it is (e.g., where the sensor is intended to be placed on a subject), the wavelength(s) of light emitted by light source 16, the intensity of light emitted by light source 16 (e.g., output wattage or Joules), the mode of light source 16 (e.g., pulsed versus CW), any other suitable information, or any combination thereof. This information may be used by monitor 14 to select appropriate algorithms, lookup tables and/or calibration coefficients stored in monitor 14 for calculating the subject's physiological parameters.

Encoder 42 may contain information specific to subject 40, such as, for example, the subject's age, weight, and diagnosis. This information about a subject's characteristics may allow monitor 14 to determine, for example, subject-specific threshold ranges in which the subject's physiological parameter measurements should fall and to enable or disable additional physiological parameter algorithms. Encoder 42 may, for instance, be a coded resistor that stores values corresponding to the type of sensor unit 12 or the type of each sensor in the sensor array, the wavelengths of light emitted by light source 16 on each sensor of the sensor array, and/or the subject's characteristics. In some embodiments, encoder 42 may include a memory on which one or more of the following information may be stored for communication to monitor 14: the type of the sensor unit 12; the wavelengths of light emitted by light source 16; the particular acoustic range that each sensor in the sensor array is monitoring; a signal threshold for each sensor in the sensor array; any other suitable information; or any combination thereof.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, a time processing unit (TPU) 58 may provide timing control signals to light drive circuitry 60, which may control the activation of light source 16. For example, TPU 58 may control pulse timing (e.g., pulse duration and inter-pulse interval) for TD-PA monitoring system. TPU 58 may also control the gating-in of signals from detector 18 through amplifier 62 and switching circuit 64. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 is filled. In some embodiments, there may be multiple separate parallel paths having components equivalent to amplifier 66, filter 68, and/or A/D converter 70 for multiple light wavelengths or spectra received. Any suitable combination of components (e.g., microprocessor 48, RAM 54, analog to digital converter 70, any other suitable component shown or not shown in FIG. 2) coupled by bus 50 or otherwise coupled (e.g., via an external bus), may be referred to as processing equipment.

In the embodiment shown, light source 16 may include modulator 44, in order to, for example, perform FD-PA analysis. Modulator 44 may be configured to provide intensity modulation, spatial modulation, any other suitable optical signal modulations, or any combination thereof. For example, light source 16 may be a CW light source, and modulator 44 may provide frequency modulation of the CW light source such as a linear frequency modulation. In some embodiments, modulator 44 may be included in light drive 60, or other suitable components of physiological monitoring system 10, or any combination thereof.

In some embodiments, microprocessor 48 may determine the subject's physiological parameters, such as blood pressure, SpO₂, SvO₂, oxy-hemoglobin concentration, deoxy-hemoglobin concentration, total hemoglobin concentration (t_{HB}), and/or pulse rate, using various algorithms and/or look-up tables based on the value of the received signals and/or data corresponding to the acoustic response received by detector 18. Signals corresponding to information about subject 40, and particularly about the acoustic signals emanating from a subject's tissue over time, may be transmitted from encoder 42 to decoder 74. These signals may include, for example, encoded information relating to subject characteristics. Decoder 74 may translate these signals to enable the microprocessor to determine the thresholds based at least in part on algorithms or lookup tables stored in ROM 52. In some embodiments, user inputs 56 may be used enter information, select one or more options, provide a response, input settings, any other suitable inputting function, or any combination thereof. User inputs 56 may be used to enter information about the subject, such as age, weight, height, diagnosis, medications, treatments, and so forth. In some embodiments, display 20 may exhibit a list of values, which may generally apply to the subject, such as, for example, age ranges or medication families, which the user may select using user inputs 56.

Calibration device 80, which may be powered by monitor 14 via a communicative coupling 82, a battery, or by a conventional power source such as a wall outlet, may include any suitable signal calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system). Reference blood pressure measurements from calibration device 80 may be accessed by microprocessor 48 for use in calibrating the blood pressure measurements.

The acoustic signal attenuated by the tissue of subject 40 can be degraded by noise, among other sources. Movement of the subject may also introduce noise and affect the signal. For example, the contact between the detector and the skin, or the light source and the skin, can be temporarily disrupted when movement causes either to move away from the skin. Another source of noise is electromagnetic coupling from other electronic instruments.

Noise (e.g., from subject movement) can degrade a sensor signal relied upon by a care provider, without the care provider's awareness. This is especially true if the monitoring of the subject is remote, the motion is too small to be observed, or the care provider is watching the instrument or other parts of the subject, and not the sensor site. Processing sensor signals may involve operations that reduce the amount of noise present in the signals, control the amount of noise present in the signal, or otherwise identify noise components in order to prevent them from affecting measurements of physiological parameters derived from the sensor signals.

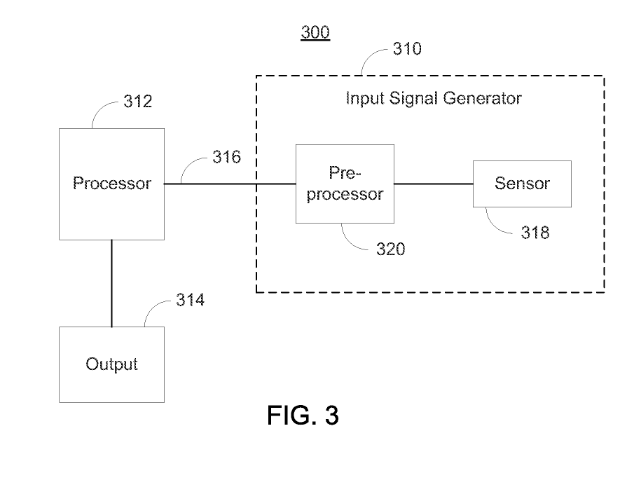

FIG. 3 is an illustrative signal processing system 300 in accordance with an embodiment that may implement the signal processing techniques described herein. In some embodiments, signal processing system 300 may be included in a physiological monitoring system (e.g., physiological monitoring system 10 of FIGS. 1-2). In the illustrated embodiment, input signal generator 310 generates an input signal 316. As illustrated, input signal generator 310 may include pre-processor 320 coupled to sensor 318, which may provide input signal 316. In some embodiments, pre-processor 320 may be a photoacoustic module and input signal 316 may be a photoacoustic signal. In an embodiment, pre-processor 320 may be any suitable signal processing device and input signal 316 may include one or more photoacoustic signals and one or more other physiological signals, such as a photoplethysmograph signal. It will be understood that input signal generator 310 may include any suitable signal source, signal generating data, signal generating equipment, or any combination thereof to produce signal 316. Signal 316 may be a single signal, or may be multiple signals transmitted over a single pathway or multiple pathways.

Pre-processor 320 may apply one or more signal processing operations to the signal generated by sensor 318. For example, pre-processor 320 may apply a pre-determined set of processing operations to the signal provided by sensor 318 to produce input signal 316 that can be appropriately interpreted by processor 312, such as performing A/D conversion. In some embodiments, A/D conversion may be performed by processor 312. Pre-processor 320 may also perform any of the following operations on the signal provided by sensor 318: reshaping the signal for transmission, multiplexing the signal, modulating the signal onto carrier signals, compressing the signal, encoding the signal, and filtering the signal.

In some embodiments, signal 316 may be coupled to processor 312. Processor 312 may be any suitable software, firmware, hardware, or combination thereof for processing signal 316. For example, processor 312 may include one or more hardware processors (e.g., integrated circuits), one or more software modules, and computer-readable media such as memory, firmware, or any combination thereof. Processor 312 may, for example, be a computer or may be one or more chips (i.e., integrated circuits). Processor 312 may, for example, include an assembly of analog electronic components. Processor 312 may calculate physiological information. For example, processor 312 may perform time domain calculations, spectral domain calculations, time-spectral transformations (e.g., fast Fourier transforms, inverse fast Fourier transforms), any other suitable calculations, or any combination thereof. Processor 312 may perform any suitable signal processing of signal 316 to filter signal 316, such as any suitable band-pass filtering, adaptive filtering, closed-loop filtering, any other suitable filtering, and/or any combination thereof. Processor 312 may also receive input signals from additional sources (not shown). For example, processor 312 may receive an input signal containing information about treatments provided to the subject. Additional input signals may be used by processor 312 in any of the calculations or operations it performs in accordance with processing system 300.

In some embodiments, all or some of pre-processor 320, processor 312, or both, may be referred to collectively as processing equipment. For example, processing equipment may be configured to amplify, filter, sample and digitize signal 316 (e.g., using an analog to digital converter), and calculate physiological information from the digitized signal.

Processor 312 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. In some embodiments, processor 312 may store physiological measurements or previously received data from signal 316 in a memory device for later retrieval. In some embodiments, processor 312 may store calculated values, such as blood pressure, pulse rate, blood oxygen saturation (e.g., arterial, venous, and/or both), hemoglobin concentration (e.g., oxygenated, deoxygenated, or total), or any other suitable calculated values, in a memory device for later retrieval. Processor 312 may be coupled to a calibration device (not shown) that may generate or receive as input reference blood pressure measurements for use in calibrating blood pressure calculations.

Processor 312 may be coupled to output 314. Output 314 may be any suitable output device such as one or more medical devices (e.g., a medical monitor that displays various physiological parameters, a medical alarm, or any other suitable medical device that either displays physiological parameters or uses the output of processor 312 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 300 may be incorporated into system 10 (FIGS. 1 and 2) in which, for example, input signal generator 310 may be implemented as part of sensor unit 12 (FIGS. 1 and 2) and monitor 14 (FIGS. 1 and 2) and processor 312 may be implemented as part of monitor 14 (FIGS. 1 and 2). In some embodiments, portions of system 300 may be configured to be portable. For example, all or part of system 300 may be embedded in a small, compact object carried with or attached to the subject (e.g., a watch, other piece of jewelry, or a smart phone). In some embodiments, a wireless transceiver (not shown) may also be included in system 300 to enable wireless communication with other components of system 10 (FIGS. 1 and 2). As such, system 10 (FIGS. 1 and 2) may be part of a fully portable and continuous physiological monitoring solution. In some embodiments, a wireless transceiver (not shown) may also be included in system 300 to enable wireless communication with other components of system 10. For example, pre-processor 320 may output signal 316 (e.g., which may be a pre-processed photoacoustic signal) over BLUETOOTH, IEEE 802.11, WiFi, WiMax, cable, satellite, Infrared, any other suitable transmission scheme, or any combination thereof. In some embodiments, a wireless transmission scheme may be used between any communicating components of system 300.

It will also be understood that while some of the equations referenced herein are continuous functions, the processing equipment may be configured to use digital or discrete forms of the equations in processing the acquired PA signal.

In some embodiments, the illuminated region of interest may include a blood vessel such as an artery, vein, or capillary, for example. The blood within the vessel may absorb a portion of the incident optical fluence at the vessel. The resulting acoustic pressure signal may exhibit two sequential peaks (in the time domain) generated primarily from the boundary between the blood and the adjacent tissue (e.g., a blood vessel). The acoustic pressure signal, as detected at a suitable detector, may be greater when that boundary surface faces the detector. The first peak may be indicative of the front boundary between the blood and the vessel (relatively closer to the light source), and the second peak may be indicative of the back boundary between the blood and the vessel (relatively further from the light source).

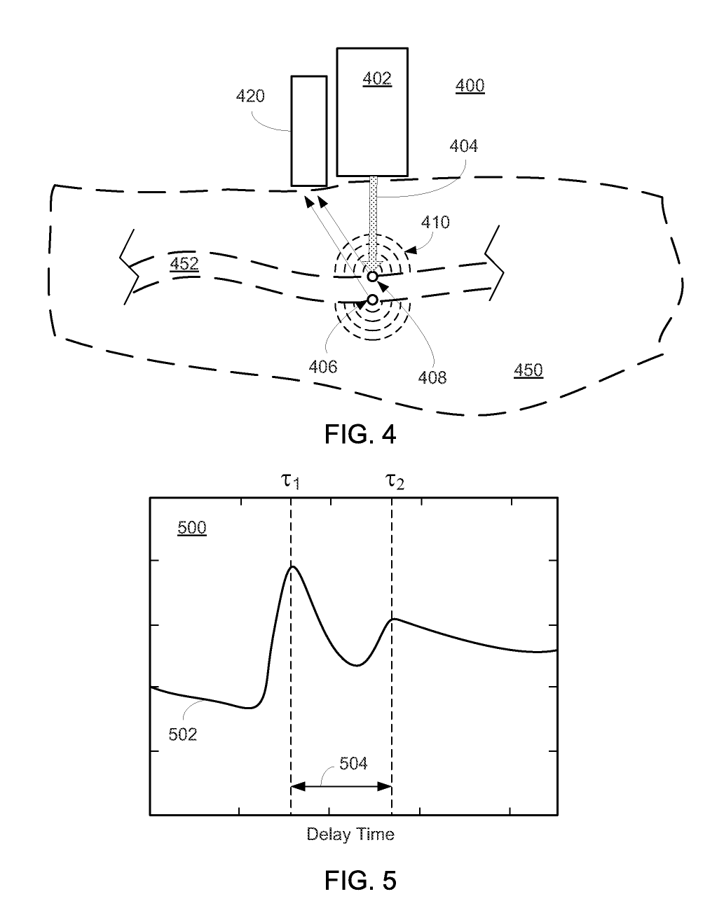

FIG. 4 is an illustrative photoacoustic arrangement 400, in accordance with some embodiments of the present disclosure. Light source 402, controlled by a suitable light drive (e.g., a light drive of system 300 or system 10, although not shown in FIG. 4), may provide photonic signal 404 to subject 450. Photonic signal 404 may be attenuated along its pathlength in subject 450 prior to reaching blood vessel 452. A constituent of the blood in blood vessel 452 such as, for example, hemoglobin, may absorb at least some of photonic signal 404. Accordingly, the blood may exhibit an acoustic pressure response via the photoacoustic effect, which may act on the boundary of blood vessel 452. Acoustic pressure signals 410 may travel through subject 450, originating substantially from the front boundary 408 and back boundary 406 of blood vessel 452. Acoustic detector 420 may detect acoustic pressure signals 410 traveling through tissue of subject 450, and output (not shown) a photoacoustic signal that may be processed. Because the path length between point 408 and acoustic detector 420 is shorter than the pathlength between point 406 and acoustic detector 420, it may be expected that acoustic pressure signals from point 408 may reach acoustic detector 420 before acoustic pressure signals from point 406. Additionally, in some arrangements, because the path length between point 408 and acoustic detector 420 is shorter than the path length between point 406 and acoustic detector 420, it may be expected that an acoustic pressure signal from point 408 may exhibit a relatively larger peak than an acoustic pressure signal from point 406. Accordingly, acoustic detector 420 may detect two sequential peaks in acoustic pressure signal 410 generated by photonic signal 404 directed at blood vessel 452.

FIG. 5 is a plot 500 of an illustrative photoacoustic signal 502, including two peaks, in accordance with some embodiments of the present disclosure. The abscissa of plot 500 is presented in units proportional to time (e.g., delay time relative to a light pulse), while the ordinate of plot 500 is presented in arbitrary units of signal intensity. At least a portion of photoacoustic signal 502 corresponds to the acoustic pressure response of blood within a blood vessel. Photoacoustic signal 502 exhibits a first peak and a second peak, located at respective times τ₁ and τ₂. The first peak corresponds to the front boundary of the blood vessel, relatively nearer to the acoustic detector. The second peak corresponds to the back boundary of the blood vessel, relatively further from the acoustic detector. It will be understood that the blood vessel diameter may also be determined by measuring other locations of the blood vessel, such as the change in the right to left sides of the blood vessels when suitable acoustic sensors are used. Time difference 504 between τ₁ and τ₂ indicates the relative difference in delay time between acoustic pressure signals from the front and back boundaries. The signal intensity may correspond to the absorption of the particular constituent of the region of interest. In some embodiments, analysis of the first and second peaks may allow the determination of one or more physiological parameters. When measuring the blood pressure in an artery, the distance between the two peaks will vary over the cardiac cycle and the different distances may correspond to different pressures.

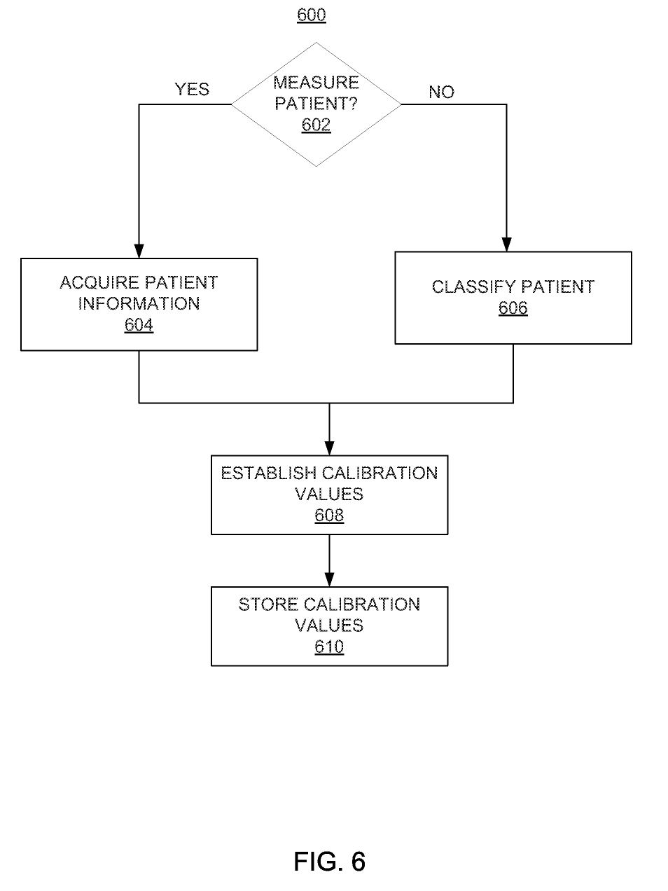

FIG. 6 is a flow diagram 600 of illustrative steps for establishing calibration values for use in determining continuous non-invasive blood pressure using photoacoustics, in accordance with some embodiments of the present invention. Calibration values may be used to establish a relationship between blood pressure and blood vessel parameters such as size. For example, calibration values may correspond to a distensibility or compliance of a blood vessel. Distensibility is the ability of a blood vessel to stretch, and is represented as the relationship between the change in the volume (ΔV) of the vessel over the volume (V) per the change in pressure (ΔV):

$$D = \frac{Δ V}{\frac{V}{Δ P}} \qquad ( 14 )$$

Compliance is the ability of a blood vessel to stretch and hold volume, and is represented as the relationship between the change in the volume (ΔV) of the vessel over the change in pressure (ΔV):

$$C = \frac{Δ V}{Δ P} \qquad ( 15 )$$

Although distensibility and compliance may change over time in a subject, e.g., as the subject ages, these parameters are generally stable over the short term. Calibration values may be established by determining the distensibility or compliance, resulting in a known relationship between blood vessel size (e.g., volume) and blood pressure for a particular subject.

At step 602 it may be determined whether the calibration values will be generated based on measurements of a subject or based on pre-stored information. Utilizing measured values of the subject to establish calibration values may provide accuracy based on the subject's current physical condition. Utilizing pre-stored information (e.g., empirical data and/or characteristics of the subject) to establish calibration values may allow for prompt determination of blood pressure using photoacoustics without the need to perform initial measurements of the subject.

If calibration values are to be established based on measured subject information, processing may proceed to step 604. Step 604 may include acquiring information from a subject that may be used to establish calibration values based on a relationship between blood vessel size and blood pressure, e.g., distensibility or compliance. It will be understood that any suitable measurement may be performed to establish a relationship between blood vessel size and blood pressure for a subject. In some embodiments, a photoacoustic system such as system 10 may allow for the measurement of the size of a region of interest such as blood vessels. The photoacoustic system may have a resolution that is sufficient to detect changes in blood vessel size that correspond to changes in blood pressure. In an exemplary embodiment a change in blood vessel diameter of 1 mm may correspond to a change in blood pressure such as 65 mmHg. A resolution for the photoacoustic system may be set such as to capture changes in blood vessel diameter such as 0.01 mm. A sample rate for the photoacoustic system may be set to acquire a sufficient number of samples to capture the full range of the change in blood vessel volume associated with changes in blood pressure. In an exemplary embodiment the sampling rate may be set to 100 to 1000 Hz.

It will be understood that the photoacoustic system may measure the size in various manners, e.g., by measuring a time difference between peaks in the photoacoustic signal, which may correspond to the distance between the walls of a blood vessel (i.e., diameter) or by generating complex images from which diameter, volume, or other parameters may be determined. In some embodiments, acquiring patient information at step 604 may include performing photoacoustic measurements of the blood vessel before, during, and/or after performing measurements of blood pressure, e.g., using a conventional blood pressure monitoring device. In some embodiments the photoacoustic measurements and blood pressure measurements may be repeated over a period of time to capture a series of values corresponding to the expansion and contraction of the blood vessel over a cardiac cycle, e.g., corresponding to the systolic and diastolic pressures. A conventional blood pressure monitoring device (e.g., an aneroid or mercury sphygmomanometer and occluding cuff) may also measure both the systolic and diastolic pressures. When the photoacoustic measurements are performed on the radial artery, the calibration may be performed using a standard brachial artery blood pressure measurement and the measurement(s) may be automatically transmitted to the photoacoustic system.

If calibration values are to be established without measured patient information, processing may proceed to step 606. Step 606 may include acquiring information about a subject from which typical relationships between variations in blood vessel size and blood pressure may be determined. For example, a subject's age, sex, height, weight, health history, blood pressure, heart rate, and any other relevant factors may be used to establish typical calibration values. The acquired information may be used in combination with pre-stored information such as empirical data or physiological models to determine the appropriate calibration values for the subject.

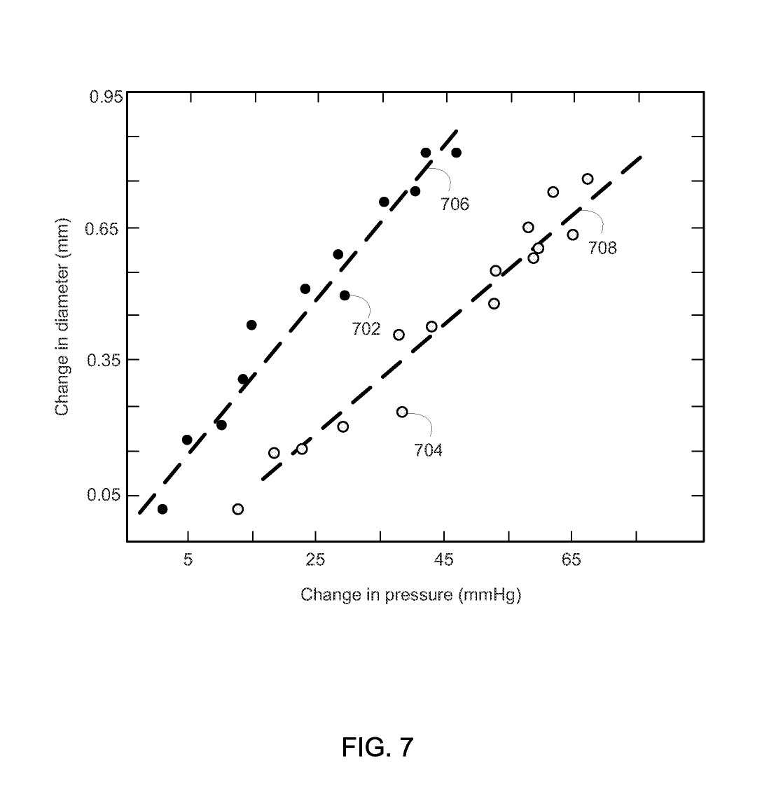

At step 608 blood pressure parameters may be determined for the subject. In some embodiments, where the subject's blood vessel is measured at step 604, a series of blood vessel size values and blood pressure values may be compared to generate a best fit to be used to establish calibration values. An exemplary embodiment of establishing calibration values is depicted in FIG. 7.

FIG. 7 depicts two sets of points corresponding to a relationship between blood vessel diameter and blood pressure. The abscissa of FIG. 7 may depict a change in blood pressure and may be in units of mmHg, while the ordinate of FIG. 7 may depict a change in diameter for a blood vessel and may be in units of mm. Points 702 may be depicted as black circles and may correspond to individual measurements of blood vessel diameter and blood pressure for a first subject, while points 704 may be depicted as white circles and may correspond to a second subject. Points 702 may be typical of a younger subject with blood vessels having a higher level of compliance and distensibility, while points 704 may be typical of an older subject with blood vessels having a lower level of compliance and distensibility. It will be understood that points 702 or 704 may be used to establish calibration points in any suitable manner, such as a line fit or curve fit algorithm. In an exemplary embodiment, line fit 706 may correspond to calibration values for the first subject, and line fit 708 may correspond to calibration values for a second subject.

Returning to step 608, in some embodiments where the subject's calibration values are to be determined based on patient information, the acquired information may be analyzed to determine calibration values to be used in determining blood pressure from blood vessel size. It will be understood that the calibration values may be established in any suitable manner based on the patient information. In some embodiments, one or more formulae may be used to establish the calibration values based on typical values for a person's age, height, weight, blood pressure, physical condition, medical history, etc.

At step 610 the established calibration values may be stored for use in determining blood pressure from blood vessel size using photoacoustics. It will be recognized that the calibration values may be stored in any suitable manner, e.g., associated with a particular subject, stored in one or more predetermined memory locations, or in any other suitable manner.

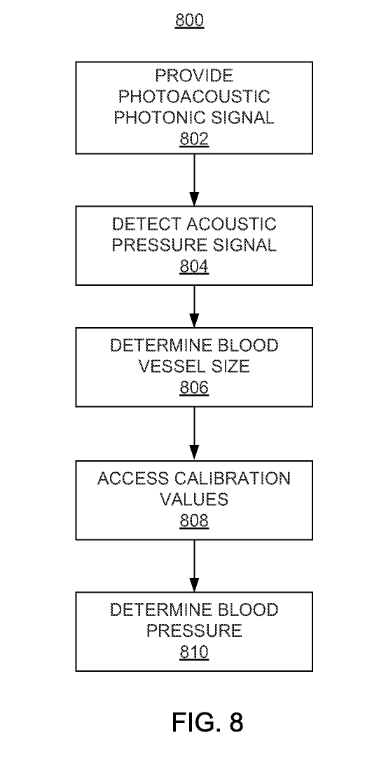

FIG. 8 is a flow diagram 800 of illustrative steps for performing continuous non-invasive blood pressure measurements using photoacoustics, in accordance with some embodiments of the present disclosure.

Step 802 may include a suitable light source (e.g., light source 16 of system 10) of system 300 providing a photonic signal to a subject. The light source may be a pulsed light source, continuous wave light source, any other suitable light source, or any combination thereof. In some embodiments, modulator 44 may be used to modulate the photonic signal of the light source. In some embodiments, the photonic signal may be focused or otherwise spatially modulated. For example, the photonic signal may be focused on a blood vessel, causing a relatively stronger photoacoustic response, and accordingly a stronger photoacoustic signal. In an exemplary embodiment a change in blood vessel diameter of 1 mm may correspond to a change in blood pressure such as 65 mmHg. A resolution for the photoacoustic system may be set such as to capture changes in blood vessel diameter such as 0.01 mm. A sample rate for the photoacoustic system may be set to acquire a sufficient number of samples to capture the full range of the change in blood vessel volume associated with changes in blood pressure. In an exemplary embodiment the sampling rate may be set to 100 to 1000 Hz.

Step 804 may include detecting an acoustic pressure signal. In some embodiments, an acoustic detector such as, for example, detector 18 or sensor 318 may detect the acoustic pressure signal. The acoustic detector may output an electrical signal to suitable processing equipment of monitor 14 or system 300. The acoustic pressure signal may be detected as a time series (e.g., in the time domain or sample number domain), as a spectral series (e.g., in the frequency domain), any other suitable series, or any combination thereof. In some embodiments, pre-processor 320 may pre-process the detected acoustic pressure signal. For example, pre-processor 320 may perform filtering, amplifying, de-multiplexing, de-modulating, sampling, smoothing, any other suitable pre-processing, or any combination thereof. Step 804 may include detecting a received acoustic pressure signal over any suitable sampling period. For example, a sampling window may be sufficient to detect a number of samples to capture the full range of motion of the blood vessel from a systolic pressure to a diastolic pressure, some portion of the range, or a series of cycles of the range. The resulting photoacoustic signal may be filtered, transformed, or otherwise modified in any suitable manner for additional processing.

Step 806 may include system 300 determining blood vessel size based on the photoacoustic signal. Blood vessel size may be determined in any suitable manner, such as determining a blood vessel diameter based on identifying peaks that correspond to blood vessel walls as described herein. The processor may use a peak finding technique to determine the first peak corresponding to a blood vessel. For example, the processor may locate a maximum in the photoacoustic signal, locate a zero in the first derivative of the photoacoustic signal, perform any other suitable peak finding technique, or any combination thereof. The peak finding technique may operate on only a subset of the photoacoustic signal. For example, the peak finding algorithm may only start looking for a peak after a predetermined time or sample number. The starting location may be determined based on the expected depth location of the blood vessel of interest. Determining a first peak may include determining a peak value, determining a peak location (e.g., in time or sample number), determining a peak property (e.g., full-width at half maximum, height to width ratio), comparing a property of a peak to a predetermined threshold (e.g., to qualify the peak), performing any other suitable determination, or any combination thereof.

Step 806 may include system 300 determining a second peak corresponding to the blood vessel using a peak finding technique. For example, processor 312 may locate a maximum in the photoacoustic signal, locate a zero in the first derivative of the photoacoustic signal, use the determined first peak to aid in locating the second peak (e.g., use a relative time value), perform any other suitable peak finding technique, or any combination thereof. Determining a second peak may include determining a peak value, determining a peak location (e.g., in time or sample number), determining a peak property (e.g., full-width at half maximum, height to width ratio), comparing a property of a peak to a predetermined threshold (e.g., to qualify the peak), performing any other suitable determination, or any combination thereof.

It will be understood that the size of the blood vessel may be measured in the time difference between the two peaks, in the number of samples between the two peaks, and/or in units of length. It will be understood that other values may be measured or calculated based on the photoacoustic signal, such as a blood vessel volume. It will also be understood that a series of blood vessel size measurements may be performed over time, such as over a given sampling window for the photoacoustic signal. The series of measurements may correspond to the change in blood pressure over a cardiac cycle or some portion thereof.

Step 808 may include system 300 accessing suitable calibration values. The calibration values may be stored in any suitable manner as described herein. In an exemplary embodiment the calibration values may be in the form of a formula or look-up table. In the case of measured calibration values, the calibration values may correspond to a particular subject, and may allow for blood pressure to be determined based on the measured blood vessel size. In the case of calibration values derived without direct subject measurements, the calibration values may correspond to a hypothetical or typical patient having similar physical characteristics to the subject, and may allow for blood pressure to be determined based on the measured blood vessel size.

Step 810 may include system 300 determining blood pressure for the subject based on the blood vessel size and the calibration values. As an example, the following equation may be used:

$$B P ( t ) = B {P}_{1} + \frac{V ( t ) - {V}_{1}}{{C}_{1}} , \qquad ( 16 )$$

where BP(t) is the determined blood pressure, BP₁ is a blood pressure calibration value, V₁ is a volume calibration value, C₁ is a compliance calibration value, and V(t) is the volume computed at step 804. BP₁ may be selected to correspond to the systolic, diastolic, or mean blood pressure measured from the subject during calibration or selected from pre-stored information based on the subject's characteristics. C₁ may be determined based on Eq. 15 using the measured calibration information or selected from pre-stored information based on the subject's characteristics. V₁ may be determined based on the measured calibration information or selected from pre-stored information based on the subject's characteristics. As another example, the following equation may also be used:

$$B P ( t ) = B {P}_{1} + ( S ( t ) - {S}_{1} ) ( \frac{Δ B {P}_{1}}{Δ {S}_{1}} ) , \qquad ( 17 )$$

where BP(t) is the determined blood pressure, BP₁ is a blood pressure calibration value (e.g., a measured diastolic value), S₁ is a size calibration value (e.g., a measured size at diastolic pressure), ΔBP₁ is a delta blood pressure calibration value (e.g., a measured difference between systolic and diastolic pressure), ΔS₁ is delta size calibration value (e.g., a measured difference between blood vessel size at systolic and diastolic pressure), and S(t) is the blood vessel size measured at step 804. It will be understood that Eqs. 16 and 17 are merely illustrative and any suitable equation and/or relationship may be used to compute blood pressure. As an example, while Eq. 16 assumes that C₁ is constant as a blood vessel changes in size, this is not necessarily the case. As a blood vessel increases in size, its compliance may decrease. Therefore, Eq. 16 may be modified such that C₁ is a function of V(t). As another example, while the blood pressure in Eq. 17 changes linearly with respect to blood vessel size, Eq. 17 may be modified such that the blood pressures scales non-linearly with respect to blood vessel size

In an exemplary embodiment a current blood pressure value may be calculated from the most recent measured blood vessel size. In another embodiment a series of values of blood vessel size representing some portion of the cardiac cycle may be used to calculate a series of blood pressure values. The determined values may correspond to the systolic blood pressure value, diastolic blood pressure value, a mean of the blood pressure values, an average of the blood pressure values, or any other suitable blood pressure calculation. Once a suitable blood pressure value has been determined the value may be displayed, transmitted, or otherwise displayed to a user.

The steps of FIG. 8 may be repeated to continuously determine the blood pressure of a subject. As discussed above, the compliance of a subject may change over time. Accordingly, when the calibration information is measured from the subject, new calibration information may be determined from time to time. For example, new calibration information may be determined periodically (e.g., every 5, 10, or 15 minutes) and/or when the blood pressure of the subject changes significantly.

The calculation of blood pressure using photoacoustics may also be combined with other techniques for determining blood pressure. For example, pulse transit time and heart rate interval are also understood to be a function of blood pressure. Pulse transit time and heart rate interval may be determined in any suitable manner, such as using photoacoustics or an electrocardiogram technique. The results determined from a plurality of different measurement techniques could be averaged or otherwise combined to determine blood pressure.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A method for determining blood pressure of a subject, the method comprising:
receiving a photoacoustic signal;
determining, using processing equipment, one or more measurements of a physical property of a region of interest of the subject based on the photoacoustic signal; and
determining, using processing equipment, blood pressure based on the one or more measurements.

2. The method of claim 1 wherein the region of interest comprises a blood vessel.

3. The method of claim 2 wherein the one or more measurements comprise one or more measurements of the size of the blood vessel.

4. The method of claim 3 wherein determining blood pressure comprises determining blood pressure based on a comparison of the size of the blood vessel to one or more calibration values.

5. The method of claim 1 wherein determining blood pressure comprises:
accessing one or more calibration values; and
performing a calculation based on the calibration values and the one or more measurements.

6. The method of claim 5 further comprising:
performing a calibration for the subject; and
generating the one or more calibration values based on the calibration.

7. The method of claim 6 wherein the one or more calibration values is based on a distensibility of a blood vessel of the subject.

8. The method of claim 6 wherein the one or more calibration values is based on a compliance of a blood vessel of the subject.

9. The method of claim 5 wherein accessing the one or more calibration values is based on one or more physical characteristics of the subject.

10. The method of claim 9 wherein the one or more physical characteristics include one or more of subject age, height, weight, physical condition and medical history.

11. A patient monitoring system comprising:
an interface configured to receive a photoacoustic signal; and
a processor configured to:
determine one or more measurements of a physical property of a region of interest of the subject based on the photoacoustic signal; and
determine blood pressure based on the one or more measurements.

12. The patient monitoring system of claim 11 wherein the region of interest comprises a blood vessel.

13. The patient monitoring system of claim 12 wherein the one or more measurements comprise one or more measurements of the size of the blood vessel.

14. The patient monitoring system of claim 13 wherein the processor is further configured to determine blood pressure based on a comparison of the size of the blood vessel to one or more calibration values.

15. The patient monitoring system of claim 11 wherein the processor is further configured to:
access one or more calibration values; and
perform a calculation based on the calibration values and the one or more measurements.

16. The patient monitoring system of claim 15 wherein the processor is further configured to:
perform a calibration for the subject; and
generate the one or more calibration values based on the calibration.

17. The patient monitoring system of claim 16 wherein the one or more calibration values is based on a distensibility of a blood vessel of the subject.

18. The patient monitoring system of claim 16 wherein the one or more calibration values is based on a compliance of a blood vessel of the subject.

19. The patient monitoring system of claim 15 wherein the processor is further configured to access the one or more calibration values based on one or more physical characteristics of the subject.

20. The patient monitoring system of claim 19 wherein the one or more physical characteristics include one or more of subject age, height, weight, physical condition, and medical history.
